(19) United States
(12) Patent Application Publication
(10) Pub. No.: US 2025/0262393 A1
(43) Pub. Date: Aug. 21, 2025

(54) SYSTEM AND APPARATUS FOR THE DELIVERY OF NEBULIZED MEDICATIONS

(71) Applicant: Yale University, New Haven, CT (US)

(72) Inventor: Peter Kahn, New Haven, CT (US)

(21) Appl. No.: 18/581,816

(22) Filed: Feb. 20, 2024

(51) Int. Cl. A61M 15/00; A61M 16/00

(52) U.S. Cl. CPC A61M 15/0085; A61M 16/0003; A61M 16/024; A61M 2016/0042; A61M 2205/3334

(57) ABSTRACT

A system for delivering nebulized medication to a patient is provided. The system includes a first nebulizer device, a second nebulizer device, a single patient interface coupled to the first nebulizer device via a first flow path and to the second nebulizer device via a second flow path, and a computing device in communication with the first and second nebulizer devices. The computing device includes memory storing instructions for execution by at least one processor for controlling the operation of the first and second nebulizer devices to deliver a first and second medication into the first and second flow paths, respectively.

### FIELD

The present technology relates generally to the field of pulmonary medicine delivery. and more particularly, to devices, systems, and methods for delivering nebulized medications to a patient.

### BACKGROUND

Currently, many medications used in the field of pulmonary medicine are delivered via the inhalational route. The use of inhalation facilitates the direct delivery of an active pharmaceutical compound to the end organ of interest (i.e., the lungs), thus maximizing on-target effects for a given dose of medication and minimizing off-target effects. Additionally, inhalation can provide a convenient method to deliver medication without requiring access to swallowing, intravenous, or subcutaneous methods of administration. Definitionally, the use of inhaled medications requires the need for appropriate delivery mechanisms. These delivery mechanisms can introduce uncertainty and human factors deviations from suggested desired use protocols. Unlike pills, liquid, or other forms of medications which are simply swallowed or injected after which time the active pharmaceutical ingredient (“API”) becomes available for therapeutic activity, inhaled therapeutics rely on the patient to inhale the API correctly using both appropriate technique and quantity of the delivered medication for this medication to have its desired effect and minimize off target or undesired effects.

To date, one of the main modalities of drug delivery for inhalational use is the nebulizer. While the nebulizer is an appealing way to deliver medication as it requires mainly passive tidal breathing on the part of the patient, nebulized medications require significant amounts of time to deliver, may expose the patient and surrounding personnel to the API, may result in imprecise dosing of medication, and traditional nebulizers are not able to accommodate all types of APIs as some may not be able to be formulated or are not available for nebulization. Additionally, not all nebulizers are portable, and they require careful cleaning after use.

What is needed, therefore, are improved devices, systems, and methods for the delivery of nebulized medication that address at least some of the problems described above.

### SUMMARY

According to an embodiment of the present technology, a system for delivering nebulized medication to a patient is provided. The system includes a first nebulizer device, a second nebulizer device, a single patient interface coupled to the first nebulizer device via a first flow path and to the second nebulizer device via a second flow path, and a computing device in communication with the first and second nebulizer devices. The computing device includes memory storing instructions for execution by at least one processor for controlling the operation of the first and second nebulizer devices to deliver a first and second medication into the first and second flow paths, respectively.

In some embodiments, the instructions include assessing a treatment plan that includes at least one nebulization protocol for the first and second medications, communicating a first cue signal to actuate the first nebulizer device to nebulize the first medication in accordance with the nebulization protocol, and communicating a second cue signal to actuate the second nebulizer device to nebulize the second medication in accordance with the nebulization protocol.

In some embodiments, the nebulization protocol includes an instruction to nebulize the first medication and the second medication substantially concurrently. The instructions for communicating a first cue signal and communicating a second cue signal are executed at least substantially simultaneously.

In some embodiments, the nebulization protocol includes an instruction to nebulize the first medication and the second medication in sequence. The instructions for communicating a first cue signal are executed prior to the step of communicating a second cue signal.

In some embodiments, a third nebulizer device is coupled to the single patient interface via a third flow path. The computing device is in communication with the third nebulizer device for controlling its operation to deliver a third medication into the third flow path. The treatment plan further includes at least one nebulization protocol for the third medication. The instructions further include communicating a third cue signal to actuate the third nebulizer device.

In some embodiments, the first, second, and third flow paths utilize a set of common components of a flow network connected to the single patient interface. The flow network includes a mixing chamber to which nebulized medications flow from each of the first, second, and third nebulizer devices and an interface feed connected to the mixing chamber and the single patient interface.

In some embodiments, an external fluid source is coupled to the flow network. The external fluid source is adapted to increase fluid flow within the flow network and/or provide an independent therapeutic benefit such as oxygen, heliox, nitrous, etc.

In some embodiments, a cleaning system is coupled to the flow network and the single patient interface and is in communication with the computing device. The instructions further include communicating a cleaning cue signal to the cleaning system to initiate a cleaning sequence for cleaning the flow network, the single patient interface, and the nebulizer devices.

In some embodiments, a flow sensor is coupled to the interface feed for measuring the flow of fluid to the single patient interface. The flow sensor is connected to the computing device.

In some embodiments, an exhalation sensor is coupled to an exhalation flow path for assessing a patient's exhaled breath. The instructions further include receiving a signal from the exhalation sensor indicating a measured level of one or more health characteristics of the patient; comparing data in the signal from the exhalation sensor to the treatment plan; and communicating one or more adjustment signals to the first, second, or third nebulizer devices, or the external fluid source to adjust one or more delivery parameters of the first, second, or third medications based on the comparison of the data from the exhalation sensor to the treatment plan.

According to another embodiment of the present technology, a computer program product for controlling the delivery of nebulized medication to a patient is provided. The computer program product includes software instructions stored on a non-transitory computer-readable medium for assessing a treatment plan that includes at least one nebulization protocol for a first and second medications to be delivered by a first nebulizer device and a second nebulizer device, respectively; communicating a first cue signal to actuate the first nebulizer device to nebulize the first medication in accordance with the nebulization protocol; and communicating a second cue signal to actuate the second nebulizer device to nebulize the second medication in accordance with the nebulization protocol.

In some embodiments, the software instructions further include receiving a signal from a flow sensor coupled to a flow network through which the first and second nebulized medication flows to a single patient interface, the signal indicating a rate of flow of fluid through the sensor; receiving a signal from an exhalation sensor coupled to an exhalation flow path for assessing a patient's exhaled breath, the signal indicating a measured level of one or more health characteristics of the patient; comparing the data from one or both of the signals from the flow sensor and the exhalation sensor to the treatment plan; and communicating one or more adjustment signals to the first or second nebulizer devices to adjust one or more delivery parameters of the first or second medications based on the comparison of the data from the flow sensor and/or the exhalation sensor to the treatment plan.

In some embodiments, the software instructions further include storing actuation data associated with the first and second nebulizer devices; storing flow sensor data and exhalation sensor data; and transmitting the stored actuation, flow sensor, and exhalation sensor data to an external system.

According to another embodiment of the present technology, a system for controlling the delivery of nebulized medication to a patient is provided. The system includes a flow sensor for coupling to a medication delivery flow network for measuring the flow of fluid to a single patient interface, an exhalation sensor for coupling to an exhalation flow path for assessing a patient's exhaled breath, and a computing device that is connectable to the flow sensor and the exhalation sensor and to a set of two or more nebulizer devices. The computing device includes memory storing instructions for execution by at least one processor for controlling the operation of the set of nebulizer devices to deliver two or more medications into the flow network.

In some embodiments, the instructions include assessing a treatment plan that includes at least one nebulization protocol for a first and a second medication, communicating a first cue signal to actuate a first nebulizer device to nebulize the first medication in accordance with the nebulization protocol, and communicating a second cue signal to actuate a second nebulizer device to nebulize the second medication in accordance with the nebulization protocol.

In some embodiments, the instructions further include receiving a signal from the flow sensor indicating rate of flow of fluid through the sensor, receiving a signal from the exhalation sensor indicating a measured level of one or more health characteristics of the patient, comparing the data from one or both of the signals from the flow sensor and the exhalation sensor to the treatment plan, and communicating one or more adjustment signals to the first or second nebulizer devices to adjust one or more delivery parameters of the first or second medications based on the comparison of the data from the flow sensor and/or the exhalation sensor to the treatment plan.

According to yet another embodiment of the present technology, a method of administering one or more nebulized medications to a patient is provided. The method includes assessing a treatment plan that includes at least one nebulization protocol for the one or more nebulized medications to be administered by one or more nebulizer devices; communicating one or more cue signals to actuate the one or more nebulizer devices to nebulize the one or more medications in accordance with the nebulization protocol; receiving a signal from a flow sensor coupled to a flow network through which the one or more nebulized medications flow to a single patient interface, the signal indicating a rate of flow of fluid through the sensor; receiving a signal from an exhalation sensor coupled to an exhalation flow path for assessing a patient's exhaled breath, the signal indicating a measured level of one or more health characteristics of the patient; comparing the data from one or both of the signals from the flow sensor and the exhalation sensor to the treatment plan; and communicating one or more adjustment signals to the one or more nebulizer devices to adjust one or more delivery parameters of the one or more nebulized medications based on the comparison of the data from the flow sensor and/or the exhalation sensor to the treatment plan.

In some embodiments, the method further includes communicating a cleaning cue signal to a cleaning system coupled to the flow network and single patient interface to initiate a cleaning sequence for cleaning the flow network, the single patient interface, and the nebulizer devices.

Further objects, aspects, features, and embodiments of the present technology will be apparent from the drawing Figures and below description.

### BRIEF DESCRIPTION OF DRAWINGS

Some embodiments of the present technology are illustrated as an example and arc not limited by the figures of the accompanying drawings, in which like references may indicate similar elements.

FIG. 1 is a functional block diagram of a system for delivering nebulized medication to a patient according to some embodiments of the present technology.

FIG. 2 is a partial detail view of the flow network of FIG. 1 according to some embodiments of the present technology.

FIG. 3 is a partial detail view of the flow network of FIG. 1 according to some embodiments of the present technology.

FIG. 4 is an isometric view of a multi-dose nebulizer device according to some embodiments of the present technology.

FIG. 5 is a flowchart of a method of administering nebulized medications to a patient according to some embodiments of the present technology.

### DETAILED DESCRIPTION

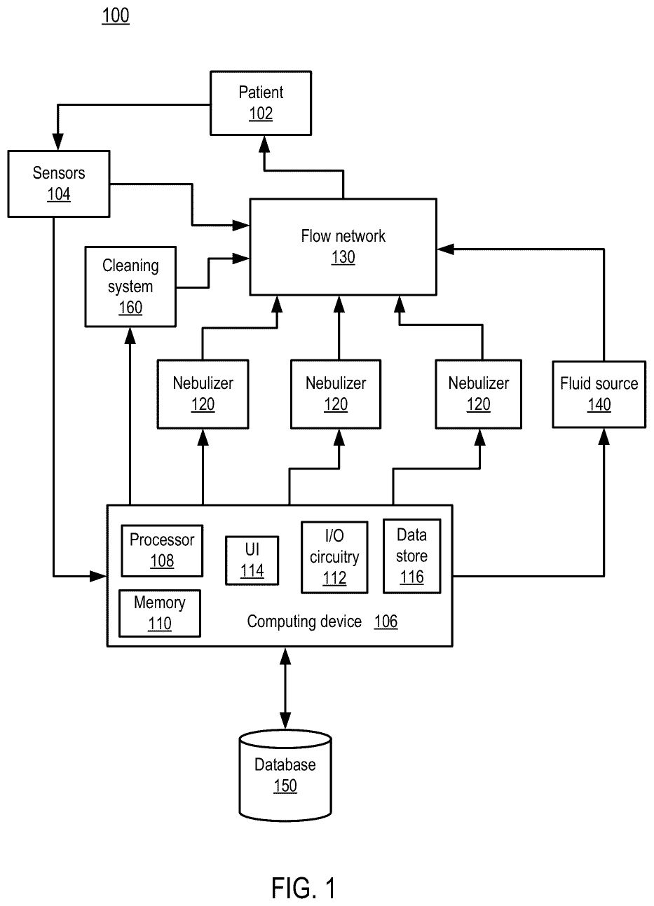

FIG. 1 illustrates a functional block diagram of a nebulized medication delivery system 100 according to some embodiments of the present technology. System 100 works with a patient 102 who desires to use at least one medication (e.g., the patient has been prescribed medication from a healthcare provider, has acquired over-the-counter medication, etc.) to be delivered via at least one nebulizer device 120 (e.g., jet nebulizers, mesh nebulizers, etc.). As used herein, the term “patient” refers to any breath-drawing organism, such as humans, dogs, cats, horses, hamsters, lizards, birds, etc. As used herein, the term “medication” refers to any substance that is designed to be nebulized and inhaled by the patient, and includes substances used for medical treatment, such as medicinal drugs, and substances used for diagnostic purposes, such as a nebulized substance that is inhaled and metabolized by the patient and then examined (e.g., examined in the patient's breath, blood, urine, feces, etc.) to diagnose the patient. The system 100 includes a first nebulizer device 120, a second nebulizer device 120, and a single patient interface 132 (i.e., the patient 102 inhales the nebulized medications delivered by the nebulizer devices 120 through only one patient interface 132) that is coupled to the first nebulizer device 120 via a first flow path FP1 and to the second nebulizer device 120 via a second flow path FP2 of a flow network 130, as discussed in more detail below. In some embodiments, a third nebulizer device 120 is coupled to the single patient interface 132 via a third flow path FP3 of the flow network 130. In some embodiments, an external fluid source 140 is coupled to the flow network 130 via a fourth flow path FP4 and is configured to augment (e.g., increase) the flow of fluid within the flow network 130. In some embodiments, at least one sensor 104 is configured to obtain respiratory behavior data of the patient 102 and to communicate the respiratory behavior data to a computing device 106.

Computing device 106 is in communication with each nebulizer device 120, sensor 104, and external fluid source 140. Computing device 106 may include, but is not limited to, a computing system (e.g., a server, a workstation computer, a desktop computer, a laptop computer, a tablet computer, an ultraportable computer, an ultramobile computer, a netbook computer and/or a subnotebook computer, etc.), a microcontroller, and/or a Smartphone. Computing device 106 includes a processor 108, a memory 110, input/output (“I/O”) circuitry 112, a user interface (“UI”) 114, and data store 116.

Processor 108 is configured to control overall operations of the computing device 106 and its associated components, such as memory 110 which stores instructions for execution by the processor 108 for controlling the operation of each nebulizer device 120 to deliver each medication into its respective flow path. I/O circuitry 112 may be configured to provide wired and/or wireless communication functionality for the computing device 106. For example, I/O circuitry 112 may be configured to receive respiratory behavior data from the sensors 104 and provide actuation and/or adjustment cue signals to the nebulizers 120 and external fluid source 140. UI 114 may include a user input device (e.g., keyboard, mouse, microphone, touch sensitive display, etc.) and/or a user output device, e.g., a display. Data store 116 may be configured to store one or more of respiratory behavior data, nebulizer requirement data, nebulizer characteristics data, historical user data, treatment plan data, etc.

The instructions stored on memory 110 for execution by the processor 108 include assessing a treatment plan that includes at least one nebulization protocol for each respective medication (i.e., a first medication to be delivered by the first nebulizer device 120, a second medication to be delivered by the second nebulizer device 120, a third medication to be delivered by the third nebulizer device 120, etc.). In some embodiments, the nebulization protocol includes the name and characteristics of each respective medication, the dose quantity of each respective medication, and nebulization parameters for delivering each respective medication, such as speed, air flow, compressed gas used to generate flow, frequency, whether to alternate the medication with other medications, etc. The instructions include communicating a first cue signal to actuate the first nebulizer device 120 to nebulize the first medication in accordance with the nebulization protocol, and communicating a second cue signal to actuate the second nebulizer device 120 to nebulizer the second medication in accordance with the nebulization protocol. In some embodiments, the nebulization protocol includes an instruction to nebulize the first medication and the second medication substantially concurrently, and the instructions for communicating a first cue signal and communicating a second cue signal are executed at least substantially simultaneously. In some embodiments, the nebulization protocol includes an instruction to nebulize the first medication and the second medication in sequence, and the instructions for communicating a first cue signal are executed prior to the step of communicating a second cue signal. In some embodiments, the instructions include communicating a third cue signal to actuate the third nebulizer device 120 to nebulize the third medication in accordance with the nebulization protocol, which may be communicated substantially concurrently, or in sequence, with one or more of the first and second actuation cue signals. Although the cue signals are described as “first,” “second,” and “third,” these descriptors are merely used to indicate that they are separate cue signals and are not intended to limit the method to any particular order. Thus, the present technology contemplates embodiments in which the cue signals occur in any order, such as the first, second, and third cue signals occurring in numerical order; first and second cue signals occurring synchronously; the third cue signal occurring before the first and/or second cue signal; the first and third cue signals occurring synchronously and the second cue signals occurring last; etc. In some embodiments, the actuation cue signals are communicated directly to the patient and/or the patient's healthcare provider and the cue signal includes an audio cue, a visual due, a haptic cue, etc., or combinations thereof. For example, in some embodiments, the cue is visual via activating a green light on the computing device that signals the patient to actuate one or more of the nebulizer devices. In other embodiments, the actuation cue signals are electronically communicated (e.g., via wired or wireless communication protocols) to an actuation mechanism of each of the nebulizer devices to automatically actuate the respective nebulizer devices.

The at least one sensor 104 includes any respiratory monitoring technology for measuring breathing that outputs an electronic signal indicative of one or more characteristics of a patient's breathing. In one embodiment, the sensor is a flow sensor that measures flow rate and volume. In other embodiments, the sensor includes invasive monitoring, non-invasive monitoring, skin monitoring, mouth and/or nose monitoring, cardiac activity monitoring, neural activity monitoring, etc., or combinations thereof. For example, in some embodiments, the sensor includes chest straps and bands that fit around the patient's chest or abdomen and measure the expansion and contraction during breathing. The chest straps and bands include digital or analog strain gauges to detect movement. The chest straps and bands can be wired or wireless. In some embodiments, the sensor includes wearable devices (e.g., Smartwatches, Fitness Trackers, etc.) that are configured to estimate respiration rates through motion detection, ballistocardiography, or photoplethysmography. In some embodiments, the sensor includes spirometers that measure the volume of air inspired and expired by the patient's lungs via pressure, flow, and/or oscillometry methods. In some embodiments, the sensor includes microphone-based systems wherein a microphone (e.g., a dedicated microphone, a microphone that is part of a Smartphone or Smart Speaker, etc.) is placed near the patient's mouth or nose to detect breath sounds. In some embodiments, the sensor includes thermal sensors placed under the patient's nose to detect the change in temperature between inhaled and exhaled air. In some embodiments, the sensor includes impedance pneumography to measure changes in electrical impedance between two electrodes places on the patient's chest to estimate respiratory activity. In some embodiments, the sensor includes capnography devices (e.g., portable or stationary) to measure the concentration of carbon dioxide in exhaled air to provide data on respiratory rate and depth. In some embodiments, the sensor includes camera-based monitoring wherein a camera (e.g., a dedicated camera, a camera that is part of a Smartphone, etc.) captures the patient's chest movements and uses image processing algorithms to calculate the respiratory rate. In some embodiments, the sensor includes radar-based or radio wave systems that use radar or radio waves to detect body movements associated with breathing. These systems may include standalone devices or be integrated into larger systems such as Smart Home Networks. In some embodiments, the sensor includes bioimpedance measurement methods that estimate lung volume by measuring the resistance of the patient's body to small amount of electric current. In some embodiments, the sensor includes any combination of the respiratory monitoring technologies discussed above.

In some embodiments, the at least one sensor 104 includes a flow sensor that is coupled to an interface feed 134 of flow network 130. The flow sensor is configured to measure the flow of fluid within the flow network 130 delivered to the single patient interface 132. The flow sensor is connected (e.g., via a wired or wireless connection) to the computing device 106. In some embodiments, the at least one sensor 104 includes an exhalation sensor that is coupled to an exhalation flow path FP5 of the flow network 130. The exhalation sensor is connected (e.g., via a wired or wireless connection) to the computing device 106. The exhalation sensor is configured to measure one or more health characteristics of the patient 102. In some embodiments, the measured health characteristics include forced expiratory volume, peak flow rates, vital capacity, fractional exhaled nitric oxide, external allergen, and infectious agents. In some embodiments, the measured health characteristics include respiratory function and gas exchange, such as oxygen saturation, carbon dioxide levels (capnography), nitric oxide levels, helium concentration, and FEV1 and other spirometry values; metabolic indicators, such as acetone, isoprene, hydrogen and methane, and ammonia; inflammatory markers, such as fractional exhaled nitric oxide, certain volatile organic compounds (“VOCs”), and interleukin markers; infectious agents, such as specific VOCs linked to bacterial or viral infections, hydrogen and methane in breath tests for gut flora imbalances, and measurement of the presence or absence of exhaled pathogens directly (e.g., bacteria, viruses, fungi, mold) or the biomarkers of these organisms; environmental and occupational exposures, such as carbon monoxide, sulfur dioxide, nitrogen dioxide, ozone, radon decay products, and VOCs like benzene, toluene, and formaldehyde; carcinogens and cancer markers, such as interleukins associated with different types of cancer and benzene and other industrial chemicals; medication levels and drug metabolism, such as ethanol, specific markers for drug metabolism, and detection of therapeutic drug levels; nutritional and dietary indicators, such as ketones and hydrogen; cardiovascular health indicators, such as nitric oxide and VOCs related to cholesterol metabolism; toxicological exposures, such as ethylene oxide, phosphine, aldehydes, and hydrocarbons; genetic and rare disorders, such as specific VOCs or gas compositions unique to certain genetic metabolic disorders; allergic and eosinophilic reactions, such as fractional exhaled nitric oxide; pulmonary disease markers, such as specific patterns of VOCs or similar markers in conditions like chronic obstructive pulmonary disease (“COPD”), asthma, or pulmonary fibrosis; liver and kidney functions, such as ammonia levels and specific VOCs indicating liver or kidney dysfunction; gastrointestinal health, such as hydrogen and methane in breath tests for conditions like IBS and SIBO; endocrine function, such as acetone and other ketones in diabetes; immunological status, such as specific inflammatory markers and VOCs; neurological conditions, such as certain VOCs linked to neurodegenerative diseases; pediatric conditions, such as specific markers relevant to metabolic or genetic disorders in children; geriatric health indicators, such as markers indicating age-related changes in metabolism or organ function; psychiatric and neurological medication monitoring, such as detection or psychotropic drug levels or metabolites; anesthetic gas monitoring, such as measurements of inhaled and exhaled anesthetic agents; hormonal levels, such as measurements of hormone metabolites indicating endocrine function; biomarkers for autoimmune diseases, such as specific VOCs or other markets indicating autoimmune activity; exercise and performance monitoring, such as changes in respiratory gases and metabolites related to physical activity; sleep apnea and sleep disorders, such as abnormal patterns in respiratory gases during sleep; and stress and emotion-related markers, such as measurements of changes in breath composition related to stress or emotional states.

In some embodiments, the computing device 106 receives a signal from the flow sensor indicating the rate of flow of fluid through the sensor and flow network. The computing device 106 receives a signal from the exhalation sensor indicating a measured level of one or more of the health characteristics of the patient discussed above. In some embodiments, the computing device 106 stores actuation data associated with each of the nebulizer devices 120 in data store 116, stores flow sensor data and exhalation sensor data in data store 116. In some embodiments, the computing device 106 transmits the stored actuation, flow sensor, and exhalation sensor data to an external system, such as database 150. In some embodiments, the stored and/or transmitted data is analyzed for adherence to the treatment plan, predicting exacerbation of the patient's underlying health issues that are being treated by the medications, disease control, improving the treatment plan including adding, removing, or adjusting the medications, changes in the patient's health characteristics discussed above, etc.

The instructions stored on memory 110 for execution by the processor 108 include comparing the data from one or both of the signals received from the flow sensor and the exhalation sensor to the treatment plan. In some embodiments, if the comparison of data to the treatment plan results in an overall performance of system 100 that is below a predetermined threshold (e.g., the rate of flow in the flow network is too low, the patient's breath pattern, depth, and/or frequency duration are inconsistent with the nebulizer devices' requirements, etc.), the instructions include communicating one or more adjustment cue signals to one or more of the nebulizer devices 120 to adjust one or more delivery parameters (e.g., power level of the nebulizer device, duration of medication delivery, adding or removing medications if anomalies are detected, adjusting the compressed gasses used to generate flow, informing the patient's healthcare provider of proposed adjustments to the treatment plan, switching the type of nebulizer device used, etc.) of the respective medications. In some embodiments, if the comparison of data to the treatment plan results in an overall performance of system 100 that is below the predetermined threshold, the instructions include communicating a cue signal to the external fluid source 140 to actuate and augment the flow of fluid in the flow network toward the patient (e.g., increase the rate of flow of fluid in the flow network, increase the amount of fluid, increase the oxygen concentration, or provide an independent therapeutic benefit such as oxygen, heliox, nitrous, etc.). In some embodiments, if the comparison of data to the treatment plan results in an overall performance of system 100 that is below the predetermined threshold after the external fluid source 140 has augmented the flow of fluid in the flow network, the instructions include communicating an adjustment cue signal to the external fluid source 140 to further augment the flow of fluid in the flow network. In some embodiments, the external fluid source actuation cue signal is communicated directly to the patient and/or the patient's healthcare provider and the cue signal includes an audio cue, a visual due, a haptic cue, etc., or combinations thereof. For example, in some embodiments, the cue is visual via activating a green light on the computing device that signals the patient to actuate the external fluid source. In other embodiments, the cue signal is electronically communicated (e.g., via wired or wireless communication protocols) to an actuation mechanism of the external fluid source to automatically actuate the external fluid source. In some embodiments, an external fluid source 140 is positioned in the flow network 130 adjacent to each nebulizer device 120 such that the flow network 130 includes a plurality of external fluid sources 140.

In some embodiments, the external fluid source 140 is a fan, a compressed gas tank, a pump (e.g., an electric pump, a pneumatic pump, a hydraulic pump, a manual pump, etc.), oscillating membranes, a chemical reactor (e.g., a device that utilizes chemical reactions to generate flow), a user chargeable bladder or canister having a handheld bellows mechanism, chemical reactions, fans, pistons, or other electronic mechanisms, or any other device or system configured to augment flow of the fluid in the flow network 130. In some embodiments, the fluid includes ambient air, oxygen, hydrogen, argon, helium, nitrogen, chlorofluorocarbon, hydroflouroalkanes, hydrofluoroolefin, any other suitable propellant, or combinations thereof. For example, in some embodiments the fluid includes ambient air enriched with some or all of the previously mentioned fluids in various mixtures. In some embodiments, a flow regulator is associated with the external fluid source 140 to manage the volume and/or speed of flow in the flow network 130. The particular design and implementation of a flow regulator varies by embodiment. For example, a flow regulator is employed in some embodiments that utilize compressed gas as the external fluid source.

In some embodiments, the system 100 includes a cleaning system 160 that is coupled to the flow network 130 and the single patient interface 132 and is in communication with the computing device 106. After the patient has finished a medication dosing session with the system 100, the computing device 106 is configured to communicate a cleaning cue signal to the cleaning system 160 to initiate a cleaning sequence for cleaning the flow network 130, the single patient interface 132, and the nebulizer devices 120. In some embodiments, the cleaning sequence includes dispersing a cleaning solution and/or a disinfecting agent that is pumped through the flow network 130 and into the single patient interface 132 for a predetermined duration. In some embodiments, the cleaning solution and/or disinfecting agent includes a washing fluid, heat and/or steam, ultraviolet light, or combinations thereof. In some embodiments, the cleaning sequence prompts a user to wash the components using various modalities such as a dishwasher, soap and water, etc. In some embodiments, a cleaning system 160 is integrated into each of the nebulizer devices 120. In some embodiments, the cleaning system 160 is separate from the nebulizer devices 120 and is coupled to the flow network via a cleaning flow path.

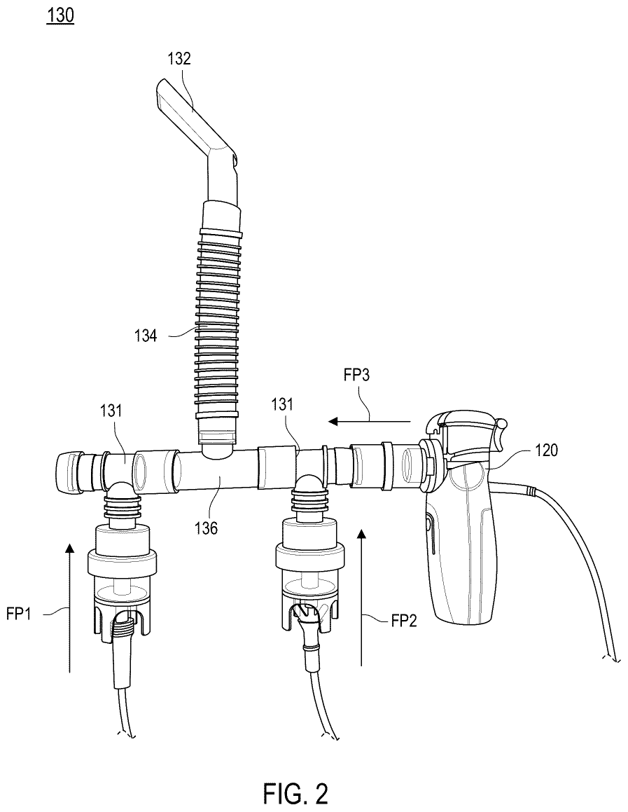

FIG. 2 illustrates an example flow network 130 as used in some embodiments of system 100. The first flow path FP1, the second flow path FP2, and the third flow path FP3 utilize a set of common components, conduits 131, to connect the nebulizer devices 120 to the single patient interface 132. The flow network 130 includes a mixing chamber 136 to which the nebulized medications flow from each nebulizer device 120. An interface feed 134 connects the mixing chamber 136 and the single patient interface 132. The single patient interface 132 can be any known respiratory connector interface, such as a mouthpiece connector, breathing mask, tracheostomy collar, etc. In the embodiment shown in FIG. 2, the first flow path FP1 connects to a first jet nebulizer device 120, the second flow path FP2 connects to a second jet nebulizer device 120, and the third flow path FP3 connects to an ultrasonic mesh nebulizer device 120. However, the present technology is not limited in this regard and contemplates embodiments having any number of the same or different types of nebulizer devices 120, such as two, three, four, five, six, etc. nebulizer devices 120. Although the mixing chamber 136 is shown as a T-junction component, the present technology is not limited in this regard and contemplates the conduits 131 being connected to the interface feed 134 via any known connection types, such as being connected in parallel, in series, via a plurality of motorized components that are actuated by the computing device 106, etc.

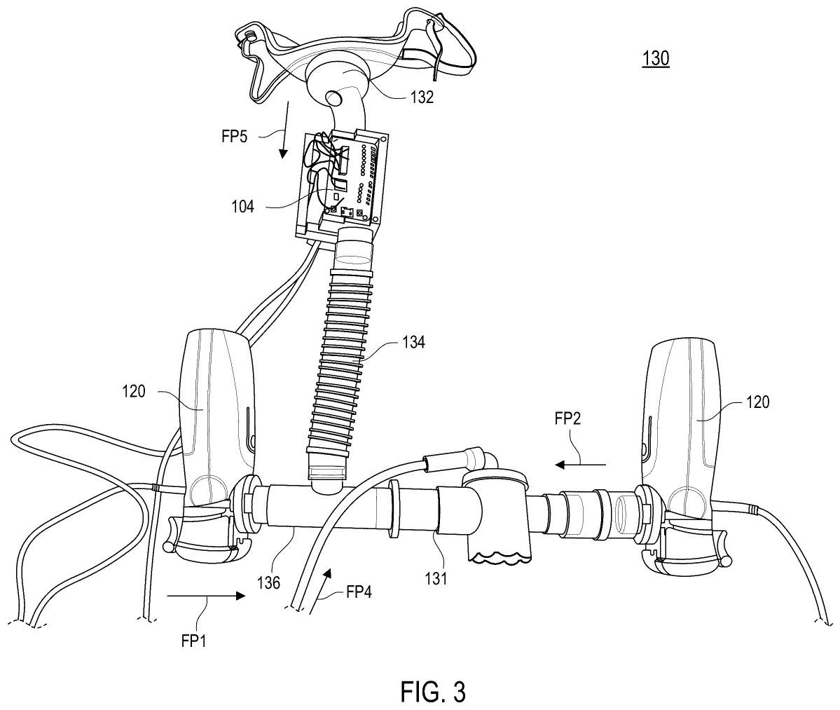

FIG. 3 illustrates an example flow network 130 as used in some embodiments of system 100. Like the embodiment shown in FIG. 2, the first flow path FP1, the second flow path FP2, and the fourth flow path FP4 utilize a set of common components, conduits 131, to connect the nebulizer devices 120 and the external fluid source 140 to the single patient interface 132. The flow network 130 includes a mixing chamber 136 to which the nebulized medications flow from each nebulizer device 120. In some embodiments, one or more of the nebulizer devices 120 are coupled directly to the mixing chamber 136. An interface feed 134 connects the mixing chamber 136 and the single patient interface 132. The single patient interface 132 can be any known respiratory connector interface, such as a mouthpiece connector, breathing mask, tracheostomy collar, etc. In the embodiment shown in FIG. 3, the first flow path FP1 connects to a first ultrasonic mesh nebulizer device 120 and the second flow path FP2 connects to a second ultrasonic mesh nebulizer device 120. However, the present technology is not limited in this regard and contemplates embodiments having any number of the same or different types of nebulizer devices 120, such as two, three, four, five, six, etc. nebulizer devices 120. The sensors 104, such as the flow sensor and exhalation sensor discussed above, are coupled to the interface feed 134 at or adjacent to the exhalation flow path FP5. In some embodiments, the flow sensor and the exhalation sensor are separately positioned in the flow network 130, such as the flow sensor being coupled to the interface feed 134 at or adjacent to the mixing chamber 136, and the exhalation sensor being coupled to the single patient interface 132 along the exhalation flow path FP5. In some embodiments, a flow sensor is positioned in the flow network 130 adjacent to each nebulizer device 120 such that the flow network 130 includes a plurality of flow sensors. Although the mixing chamber 136 is shown as a T-junction component, the present technology is not limited in this regard and contemplates the conduits 131 being connected to the interface feed 134 via any known connection types, such as being connected in parallel, in series, via a plurality of motorized components that are actuated by the computing device 106, etc.

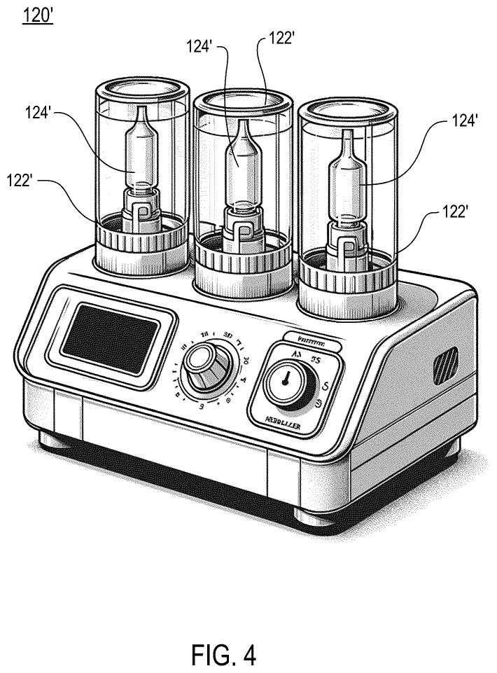

In some embodiments, one or more of the nebulizer devices 120 used in system 100 form a multi-dose nebulizer device 120′, as shown in FIG. 4. The multi-dose nebulizer device 120′ is a jet nebulizer and includes a plurality of nebulization chambers 122′ that are each configured to house and dispense a respective medication (from e.g., medication vials 124′). The nebulization chambers 122′ can be actuated substantially concurrently, in sequence, or in any combination in accordance with the nebulization protocol. Each nebulization chamber 122′ is connectable to a respective flow path FP1, FP2, FP3 for delivering the medication to the flow network 130 of system 100. Although the multi-dose nebulizer device 120′ shown in FIG. 4 includes three nebulization chambers 122′, the present technology is not limited in this regard and contemplates embodiments where the multi-dose nebulizer device 120′ includes any number of nebulization chambers 122′ greater than one, such as two, four, five, six, etc. In some embodiments, the computing device 106 is integrated into the multi-dose nebulizer device 120′.

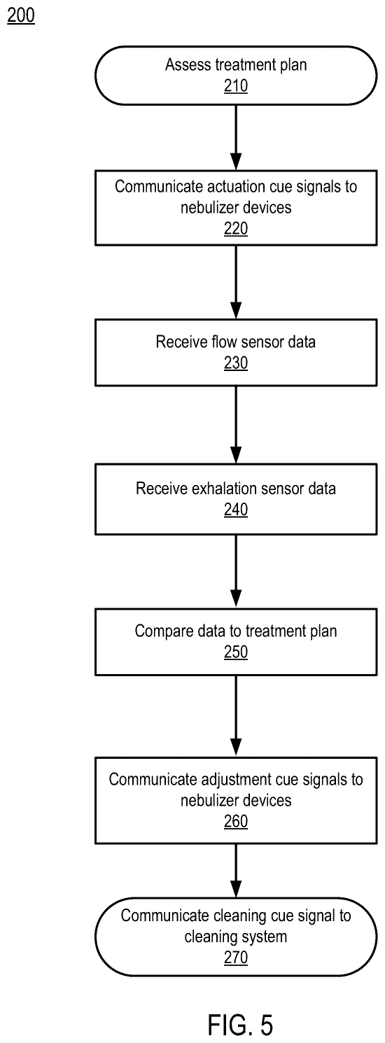

FIG. 5 illustrates a flowchart of a method 200 of administering one or more nebulized medications to a patient according to some embodiments of the present technology. At 210, the method 200 includes assessing the treatment plan that includes the at least one nebulization protocol for the one or more nebulized medications to be administered by the one or more nebulizer devices 120. At 220, the method 200 includes communicating the one or more cue signals to actuate the one or more nebulizer devices 120 to nebulize the one or more medications in accordance with the nebulization protocol. At 230, the method 200 includes receiving a signal from the flow sensor indicates the rate of flow of fluid through the sensor, as discussed above regarding system 100. At 240, the method 200 includes receiving a signal from the exhalation sensor indicating a measured level of one or more health characteristic of the patient, as discussed above regarding system 100. In some embodiments, the method 200 includes storing actuation data associated with the one or more nebulizer devices 120, storing the flow sensor data and the exhalation data, and transmitting the stored actuation, flow sensor, and exhalation sensor data to an external system, as discussed above regarding system 100. At 250, the method includes comparing the data from one or both of the signals from the flow sensor and the exhalation sensor to the treatment plan, as discussed above regarding system 100. At 260, the method 200 includes communicating one or more adjustment signals to the one or more nebulizer devices 120 to adjust one or more delivery parameters of the one or more nebulized medications based on the comparison of the data from the flow sensor and/or the exhalation sensor to the treatment plan, as discussed above regarding system 100. In some embodiments, at 270, the method 200 includes communicating a cleaning cue signal to the cleaning system 160 to initiate a cleaning sequence for cleaning the flow network 130 and the single patient interface 132, as discussed above regarding system 100.

As will be apparent to those skilled in the art, various modifications, adaptations, and variations of the foregoing specific disclosure can be made without departing from the scope of the technology claimed herein. The various features and elements of the technology described herein may be combined in a manner different than the specific examples described or claimed herein without departing from the scope of the technology. In other words, any element or feature may be combined with any other element or feature in different embodiments, unless there is an obvious or inherent incompatibility between the two, or it is specifically excluded.

References in the specification to “one embodiment,” “an embodiment,” etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition, or step being referred to is an optional (not required) feature of the technology.

The term “and/or” means any one of the items, any combination of the items, or all of the items with which this term is associated. The phrase “one or more” is readily understood by one of skill in the art, particularly when read in context of its usage.

Each numerical or measured value in this specification is modified by the term “about.” The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents of carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third, and upper third, etc.

As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the technology encompasses not only the entire group listed as a whole, but each member of the group individually and all possible subgroups of the main group. Additionally, for all purposes, the technology encompasses not only the main group, but also the main group absent one or more of the group members. The technology therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

1. A system for delivering nebulized medication to a patient, comprising:
a first nebulizer device;
a second nebulizer device;
a single patient interface, coupled to the first nebulizer device via a first flow path and to the second nebulizer device via a second flow path;
a computing device in communication with the first and second nebulizer devices and comprising memory storing instructions for execution by at least one processor for:
assessing a treatment plan including at least one nebulization protocol for the first and second medications;
controlling the operation of the first and second nebulizer devices to deliver a first and second medication into the first and second flow paths, respectively;
receiving a signal from the flow sensor indicating a rate of flow of fluid through the sensor;
receiving a signal from the exhalation sensor indicating a measured level of one or more health characteristics of the patient;
comparing data from one or both of the signals from the flow sensor and the exhalation sensor to the treatment plan; and
communicating one or more adjustment signals to the first or second nebulizer devices to adjust one or more delivery parameters of the first or second medications based on the comparison of the data from the flow sensor and/or the exhalation sensor to the treatment plan;
a flow sensor coupled to the interface feed for measuring the flow of fluid to the single patient interface and connected to the computing device; and
an exhalation sensor coupled to an exhalation flow path for assessing a patient's exhaled breath.

2. The system of claim 1, wherein the instructions comprise:
communicating a first cue signal to actuate the first nebulizer device to nebulize the first medication in accordance with the nebulization protocol; and
communicating a second cue signal to actuate the second nebulizer device to nebulize the second medication in accordance with the nebulization protocol.

3. The system of claim 2, wherein the nebulization protocol includes an instruction to nebulize the first medication and the second medication substantially concurrently, and wherein the instructions for communicating a first cue signal and communicating a second cue signal are executed at least substantially simultaneously.

4. The system of claim 2, wherein the nebulization protocol includes an instruction to nebulize the first medication and the second medication in sequence, and wherein the instructions for communicating a first cue signal are executed prior to the step of communicating a second cue signal.

5. The system of claim 2, further comprising a third nebulizer device coupled to the single patient interface via a third flow path and wherein the computing device is in communication with the third nebulizer device for controlling its operation to deliver a third medication into the third flow path;
wherein the treatment plan further includes at least one nebulization protocol for the third medication; and
wherein the instructions further comprise communicating a third cue signal to actuate the third nebulizer device.

6. The system of claim 5, wherein the first, second, and third flow paths utilize a set of common components of a flow network connected to the single patient interface, wherein the flow network comprises a mixing chamber to which nebulized medications flow from each of the first, second, and third nebulizer devices and an interface feed connected to the mixing chamber and the single patient interface.

7. The system of claim 6, further comprising an external fluid source coupled to the flow network adapted to increase fluid flow within the flow network.

8. The system of claim 6, further comprising a cleaning system coupled to the flow network and single patient interface and in communication with the computing device, comprising a cleaning solution and/or disinfecting agent, and wherein the instructions further comprise communicating a cleaning cue signal to the cleaning system to initiate a cleaning sequence for cleaning the flow network and single patient interface.

9. (canceled)

10. The system of claim 6, wherein the instructions further comprise:
communicating one or more adjustment signals to the first, second, or third nebulizer devices to adjust one or more delivery parameters of the first, second, or third medications based on the comparison of the data from the exhalation sensor to the treatment plan.

11. A computer program product for controlling delivery of nebulized medication to a patient, comprising software instructions stored on a non-transitory computer-readable medium for:
assessing a treatment plan including at least one nebulization protocol for a first and second medications to be delivered by a first nebulizer device and a second nebulizer device, respectively;
communicating a first cue signal to actuate the first nebulizer device to nebulize the first medication in accordance with the nebulization protocol;
communicating a second cue signal to actuate the second nebulizer device to nebulize the second medication in accordance with the nebulization protocol;
receiving a signal from a flow sensor coupled to a flow network through which the first and second nebulized medication flows to a single patient interface, the signal indicating a rate of flow of fluid through the sensor;
receiving a signal from an exhalation sensor coupled to an exhalation flow path for assessing a patient's exhaled breath, the signal indicating a measured level of one or more health characteristics of the patient;
comparing the data from one or both of the signals from the flow sensor and the exhalation sensor to the treatment plan; and
communicating one or more adjustment signals to the first or second nebulizer devices to adjust one or more delivery parameters of the first or second medications based on the comparison of the data from the flow sensor and/or the exhalation sensor to the treatment plan.

12. (canceled)

13. The product of claim 11, wherein the nebulization protocol includes an instruction to nebulize the first medication and the second medication substantially concurrently, and wherein the instructions for communicating a first cue signal and communicating a second cue signal are executed at least substantially simultaneously.

14. The product of claim 11, wherein the nebulization protocol includes an instruction to nebulize the first medication and the second medication in sequence, and wherein the instructions for communicating a first cue signal are executed prior to the step of communicating a second cue signal.

15. The product of claim 12, further comprising software instructions for:
storing actuation data associated with the first and second nebulizer devices;
storing flow sensor data and exhalation sensor data; and
transmitting the stored actuation, flow sensor, and exhalation sensor data to an external system.

16. A system for controlling delivery of nebulized medication to a patient, comprising:
a flow sensor, for coupling to a medication delivery flow network for measuring the flow of fluid to a single patient interface;
an exhalation sensor, for coupling to an exhalation flow path for assessing a patient's exhaled breath; and
a computing device, connectable to the flow sensor and the exhalation sensor and to a set of two or more nebulizer devices, the computing device comprising memory storing instructions for execution by at least one processor for:
controlling the operation of the set of nebulizer devices to deliver two or more medications into the flow network
assessing a treatment plan including at least one nebulization protocol for a first and a second medication;
communicating a first cue signal to actuate a first nebulizer device to nebulize the first medication in accordance with the nebulization protocol;
communicating a second cue signal to actuate a second nebulizer device to nebulize the second medication in accordance with the nebulization protocol
receiving a signal from the flow sensor indicating rate of flow of fluid through the sensor;
receiving a signal from the exhalation sensor indicating a measured level of one or more health characteristics of the patient;
comparing the data from one or both of the signals from the flow sensor and the exhalation sensor to the treatment plan; and
communicating one or more adjustment signals to the first or second nebulizer devices to adjust one or more delivery parameters of the first or second medications based on the comparison of the data from the flow sensor and/or the exhalation sensor to the treatment plan.

17. (canceled)

18. The system of claim 15, wherein the nebulization protocol includes an instruction to nebulize the first medication and the second medication substantially concurrently, and wherein the instructions for communicating a first cue signal and communicating a second cue signal are executed at least substantially simultaneously.

19. The system of claim 15, wherein the nebulization protocol includes an instruction to nebulize the first medication and the second medication in sequence, and wherein the instructions for communicating a first cue signal are executed prior to the step of communicating a second cue signal.

20. (canceled)

21. A method of administering one or more nebulized medications to a patient, comprising the steps of:
assessing a treatment plan including at least one nebulization protocol for the one or more nebulized medications to be administered by one or more nebulizer devices;
communicating one or more cue signals to actuate the one or more nebulizer devices to nebulize the one or more medications in accordance with the nebulization protocol;
receiving a signal from a flow sensor coupled to a flow network through which the one or more nebulized medications flow to a single patient interface, the signal indicating a rate of flow of fluid through the sensor;
receiving a signal from an exhalation sensor coupled to an exhalation flow path for assessing a patient's exhaled breath, the signal indicating a measured level of one or more health characteristics of the patient;
comparing the data from one or both of the signals from the flow sensor and the exhalation sensor to the treatment plan;
communicating one or more adjustment signals to the one or more nebulizer devices to adjust one or more delivery parameters of the one or more nebulized medications based on the comparison of the data from the flow sensor and/or the exhalation sensor to the treatment plan; and
communicating a cleaning cue signal to a cleaning system coupled to the flow network and single patient interface comprising a cleaning solution and/or disinfecting agent, to initiate a cleaning sequence for cleaning the flow network and single patient interface.

22. (canceled)

23. The method of claim 21, further comprising:
storing actuation data associated with the one or more nebulizer devices;
storing flow sensor data and exhalation sensor data; and
transmitting the stored actuation, flow sensor, and exhalation sensor data to an external system.

24. The product of claim 11, further comprising software instructions for communicating a cleaning cue signal to a cleaning system coupled to the flow network and single patient interface comprising a cleaning solution and/or disinfecting agent, to initiate a cleaning sequence for cleaning the flow network and single patient interface.

25. The system of claim 16, further comprising a cleaning system coupled to the flow network and single patient interface and in communication with the computing device comprising a cleaning solution and/or disinfecting agent, and wherein the instructions further comprise communicating a cleaning cue signal to the cleaning system to initiate a cleaning sequence for cleaning the flow network and single patient interface.

26. The system of claim 1, further comprising that the single patient interface consists of a mouthpiece connector.

27. The system of claim 16, further comprising that the single patient interface consists of a mouthpiece connector.
